(19) United States
(12) Patent Application Publication
(10) Pub. No.: US 2006/0246057 A1
(43) Pub. Date: Nov. 2, 2006

(54) Treatment or prevention of damage due to radiation exposure

(75) Inventor: Allan L. Goldstein, Washington, DC (US)

Correspondence Address: ROTHWELL, FIGG, ERNST & MANBECK, P.C., 1425 K STREET, N.W., SUITE 800, WASHINGTON, DC, 20005, US

(73) Assignee: RegeneRx Biopharmaceuticals, Inc, Bethesda, MD (US)

(21) Appl. No.: 10/564,766

(22) PCT Filed: Jul. 19, 2004
(86) PCT No.: PCT/US04/23075

§ 371 Date: Jun. 27, 2006

Related U.S. Application Data
(60) Provisional application No. 60/488,097, filed on Jul. 18, 2003.

(51) Int. Cl. A61K 39/395; A61K 48/00; A61K 38/08

(52) U.S. Cl. 424/133.1; 514/16; 514/17; 514/44

(57) ABSTRACT

A method of treatment or prevention of damage due to ionizing radiation exposure involves administering to a subject in need of such treatment an effective amount of a composition containing 1) a compound including a radiation damage-inhibiting polypeptide containing amino acid sequence LKKTET (such as Thymosin β4), a conservative variant of LKKTET, an actin-sequestering agent, an anti-inflammatory agent; 2) an agent which stimulates production of the compound in the subject; 3) an agent which regulates the compound in the subject; or 4) an antagonist of the compound, so as to inhibit radiation damage in the subject.

### CROSS-REFERENCE TO RELATED APPLICATION

The present application claims the benefit of U.S. Provisional Application Ser. No. 60/488,097, filed Jul. 18, 2003.

### BACKGROUND OF THE INVENTION

1. Field of the Invention

The present invention relates to the field of the treatment or prevention of damage due to radiation.

2. Description of the Background Art

For decades, ionizing radiation has frequently been used as a modality for the treatment of many types of cancers and tissue abnormalities. Although control of the delivery of such radiation has improved, the fact that it cannot be precisely controlled in many areas of the body confers certain unwanted biological side effects, including the destruction of healthy tissue, radiation burns and sickness, and other similar damage such as disruption of tissue and cellular architecture, structural changes in cytoskeletal organization and disruption of the structural organization of actin and various degenerative, immunological, and other injuries to the blood, blood vessels, microvasculatures, healthy tissues and organs secondary to radiation therapy. In particular the efficacy of therapy in cancer patients and other patients receiving radiation treatments is currently limited by the significant damage to surrounding healthy tissues which includes increased inflammatory responses and the release of toxic intermediates including inflammatory cytokines, chemokines, eicosanoids and metabolites that limit the effective dose of ionizing radiation in patients.

Radiation from other sources, including sunlight, gamma rays, X-rays, nuclear equipment, nuclear facilities, nuclear bombs, “dirty” bombs, high voltage electrical current, etc., can cause damage, sometimes severe, to tissues of exposed subjects.

There remains a need in the art for improved methods and compositions for treating or preventing the damage caused by radiation exposure.

### SUMMARY OF THE INVENTION

In accordance with the present invention, a method of treatment or prevention of damage due to radiation exposure comprising administering to a subject in need of such treatment an effective amount of a composition comprising 1) a compound including a radiation damage-inhibiting polypeptide comprising amino acid sequence LKKTET, a conservative variant of LKKTET, an actin-sequestering agent, an anti-inflammatory agent; 2) an agent which stimulates production of said compound in said subject; 3) an agent which regulates said compound in said subject; or 4) an antagonist of said compound, so as to inhibit radiation damage in said subject.

### DETAILED DESCRIPTION OF THE INVENTION

In accordance with one embodiment, the present invention relates generally to the treatment, prevention or reversal of physical, cognitive, and biological injuries resulting from exposure to ionizing radiation by the use of the peptide, Thymosin beta 4 (Thymosin β4 or Tβ4), or fragments of Tβ4 such as LKKTET, or conservative variants thereof. Sometimes these are referred to as LKKTET peptides or polypeptides. Included are N- or C-terminal variants such as KLKKTET and LKKTETQ.

Over 50% of all cancer patients receive radiation therapy to reduce tumor size. The efficacy of radiotherapy is dose limiting due to the toxic side effects of radiation and the disruption of normal tissue architecture and inflammatory, degenerative and immunological effects to surrounding tissues due either to the direct effects of the x-rays or gamma-rays or to side effects resulting from the release of toxic amounts of tissue and cellular debris from the tumors. As up to 10% of the total protein in tumors is actin and 50% of this protein is sequestered in its monomeric form when the G-actin is released into the blood following destruction of tumor tissues, the physico-chemical properties of the blood induces the polymerization of the G-actin into F-actin, the fibril form of this molecule. This flood of F-actin overwhelms the actin-sequestering properties of the blood and can result in severe pathologies. F-actin alone, when administered to experimental animals, has significant toxicity and is thought to play role in the multi-organ failure, ARDS and other syndromes associated with septic shock. A number of tissues such as the stem cells of the bone marrow, the lymphoid tissues such as the spleen and lymph nodes and the endothelial cells of the gut, have long been known to be highly sensitive to the deleterious effects of ionizing radiation. The deleterious effects on these tissues have previously been attributed to either direct or indirect effects due to the release of adrenal cortical steroids or to a variety of other additional hormones and growth factors. In addition, the structural disorganization of actin due to direct or indirect effects of radiation is thought to contribute significantly to the toxicities observed. Some of the growth factors which include inflammatory cytokines and chemokines and other agents such as eicosinoids may contribute significantly to the side effects and current limitations of radiotherapy. Tβ4, analogs and isoforms and other derivatives, by virtue of their unique properties when administered systemically, locally or topically, are effective in reducing the toxic side effects of radiotherapy. Furthermore, the unique properties of Tβ4 include radio-protective effects, thus allowing increased effective doses of radiation therapy. The invention also is applicable to treatment or prevention of damage due to radiation from other sources, including sunlight, x-rays, gamma rays, nuclear equipment, nuclear facilities, nuclear bombs, “dirty” bombs, high voltage electrical current and other sources of radiation.

Without being bound to any particular theory, it is believed that the present invention is based on the discovery that anti-inflammatory peptides and actin-sequestering peptides such as Tβ4 and a number of other actin-sequestering peptides which contain the actin binding motif and amino acid sequence LKKTET, are useful for the treatment or prevention of certain biological processes which occur due to exposure to ionizing radiation, and promote treatment or prevention of damage due to ionizing radiation exposure. These peptides have the capacity to promote repair and healing by having the ability to induce terminal deoxynucleotidyl transferase (a non-template directed DNA polymerase), to decrease the levels of one or more inflammatory cytokines and chemokines and to act as a chemotactic and angiogenic factors for endothelial cells, and thus prevent and/or heal and reverse effects that occur due to a number of factors, including exposure to certain x-rays, gamma-rays or other forms of ionizing radiation and radiotherapy of (i) cancer patients, (ii) patients receiving radiation or photo-therapy for skin disorders, or (iii) individuals exposed to acute or lethal doses of ionizing radiation. Tβ4 may act as a “rescue molecule”, preventing permanent polymerization of actin, preserving the function of actin in cells exposed to radiation and protecting the ability of normal cells to divide. Tβ4 may inhibit induction of enzymes which induce apoptosis, thereby inhibiting induction of apoptosis of normal cells which may be caused by radiation. Tβ4 may also prevent damage to tissue by modulation of transcription factors associated with improved survival of tissue. Tβ4 forms a functional ternary complex with LIM domain protein PINCH and Integrin Linked Kinase (ILK), which are essential for cell survival. Tβ4 exposure results in induction, altered localization and activation of ILK. Formation of a Tβ4-PINCH-ILK complex in cells may mediate the protection and/or repair effects of Tβ4 independently of actin polymerization. Additionally, Tβ4 stimulates the production of laminin-5 in cells which may protect, or facilitate repair of, tissue.

Tβ4 was initially identified as a protein that is up-regulated during endothelial cell migration and differentiation in vitro. Tβ4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

In accordance with one embodiment, the invention is a method of treatment or prevention of damage due to ionizing radiation exposure comprising administering to a subject in need of such treatment an effective amount of a composition comprising a radiation damage-inhibiting polypeptide comprising LKKTET, or a conservative variant is thereof having radiation damage-inhibiting activity, preferably Tβ4, an isoform of Tβ4, oxidized Tβ4, Tβ4 sulfoxide, or an antagonist of Tβ4. Administration can be before, during or after exposure of the subject to radiation, so as to protect tissue and prevent damage, and/or salvage and repair tissue.

Preferred compositions which may be used in accordance with the present invention comprise amino acid sequence LKKTET, amino acid sequence KLKKTET or LKKTETQ, Tβ4, an N-terminal variant of Tβ4, a C-terminal variant of Tβ4, an isoform of Tβ4, a splice-variant of Tβ4, oxidized Tβ4, Tβ4 sulfoxide, lymphoid Tβ4, pegylated Tβ4 or any other actin sequestering or bundling proteins having actin binding domains, or peptide fragments comprising or consisting essentially of the amino acid sequence LKKTET or conservative variants thereof, having radiation damage-inhibiting activity. International Application Serial No. PCT/US99/17282, incorporated herein by reference, discloses isoforms of Tβ4 which may be useful in accordance with the present invention as well as amino acid sequence LKKTET and conservative variants thereof, which may be utilized with the present invention. International Application Serial No. PCT/GB99/00833 (WO 99/49883), incorporated herein by reference, discloses oxidized Tβ4 which may be utilized in accordance with the present invention. Although the present invention is described primarily hereinafter with respect to Tβ4 and Tβ4 isoforms, it is to be understood that the following description is intended to be equally applicable to amino acid sequence LKKTET, KLKKTET, LKKTETQ, peptides and fragments comprising or consisting essentially of LKKTET, KLKTET or LKKTETQ, conservative variants thereof, as well as oxidized Tβ4 and Tβ4 sulfoxide, having radiation damage-inhibiting activity.

In one embodiment, the invention provides a method for healing radiation damage in a subject by contacting an area to be treated with an effective amount of a radiation damage-inhibiting composition which contains Tβ4 or a Tβ4 isoform. The contacting may be topically, systemically, enterally, by pulmonary delivery, etc. Examples of topical administration include, for example, contacting the skin with a lotion, salve, gel, cream, paste, spray, suspension, dispersion, hydrogel, ointment, or oil comprising Tβ4, alone or in combination with at least one agent that enhances Tβ4 penetration, or delays or slows release of Tβ4 peptides into the area to be treated. Systemic administration includes, for example, intravenous, intraperitoneal, intramuscular or subcutaneous injections, or inhalation, transdermal or oral administration of a composition containing Tβ4 or a Tβ4 isoform, etc. Enteral administration may include oral or rectal administration. A subject may be a mammal, is preferably human.

Tβ4, or its analogues, isoforms or derivatives, may be administered in any effective amount. For example, Tβ4 may be administered in dosages within the range of about 0.1-50 micrograms of Tβ4, more preferably in amounts of about 1-25 micrograms.

A composition in accordance with the present invention can be administered daily, every other day, etc., with a single administration or multiple administrations per day of administration, such as applications 2, 3, 4 or more times per day of administration.

Tβ4 isoforms have been identified and have about 70%, or about 75%, or about 80% or more homology to the known amino acid sequence of Tβ4. Such isoforms include, for example, Tβ4^{ala}, Tβ9, Tβ10, Tβ11, Tβ12, Tβ13, Tβ14 and Tβ15. Similar to Tβ4, the Tβ10 and Tβ15 isoforms have been shown to sequester actin. Tβ4, Tβ10 and Tβ15, as well as these other isoforms share an amino acid sequence, LKKTET, that appears to be involved in mediating actin sequestration or binding. Although not wishing to be bound to any particular theory, the activity of Tβ4 isoforms may be due, in part, to the ability to regulate the polymerization of actin. For example, Tβ4 can modulate actin polymerization in skin (e.g. β-thymosins appear to depolymerize F-actin by sequestering free G/actin). Tβ4's ability to modulate actin polymerization may therefore be due to all, or in part, its ability to bind to or sequester actin via the LKKTET sequence. Thus, as with Tβ4, other proteins which bind or sequester actin, or modulate actin polymerization, including Tβ4 isoforms having the amino acid sequence LKKTET, are likely to prevent or reduce radiation damage alone or in a combination with Tβ4, as set forth herein.

Thus, it is specifically contemplated that known Tβ4 isoforms, such as Tβ4^{ala}, Tβ9, Tβ10, Tβ11, Tβ12, Tβ13, Tβ14 and Tβ15, as well as Tβ4 isoforms not yet identified, will be useful in the methods of the invention. As such Tβ4 isoforms are useful in the methods of the invention, including the methods practiced in a subject. The invention therefore further provides pharmaceutical compositions comprising Tβ4, as well as Tβ4 isoforms Tβ4^{ala}, Tβ9, Tβ10, Tβ11, Tβ12, Tβ13, Tβ14 and Tβ15, and a pharmaceutically acceptable carrier.

In addition, other proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET, for example, can similarly be employed in the methods of the invention. Such proteins include gelsolin, vitamin D binding protein (DBP), profilin, cofilin, adsevertin, propomyosin, fincilin, depactin, DnaseI, vilin, fragmin, severin, capping protein, β-actinin and acumentin, for example. As such methods include those practiced in a subject, the invention further provides pharmaceutical compositions comprising gelsolin, vitamin D binding protein (DBP), profilin, cofilin, depactin, DnaseI, vilin, fragmin, severin, capping protein, β-actinin and acumentin as set forth herein. Thus, the invention includes the use of a radiation damage-inhibiting polypeptide comprising the amino acid sequence LKKTET (which may be within its primary amino acid sequence) and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

Tβ4 has been localized to a number of tissue and cell types and thus, agents which stimulate the production of Tβ4 can be added to or comprise a composition to effect Tβ4 production from a tissue and/or a cell. Such agents include members of the family of growth factors, such as insulin-like growth factor (IGF-1), platelet derived growth factor (PDGF), epidermal growth factor (EGF), transforming growth factor beta (TGF-β), basic fibroblast growth factor (bFGF), thymosin α1 (Tα1) and vascular endothelial growth factor (VEGF). More preferably, the agent is transforming growth factor beta (TGF-β) or other members of the TGF-β superfamily. Tβ4 compositions of the invention may reduce certain effects of radiation by effectuating growth of the connective tissue through extracellular matrix deposition, cellular migration and vascularization.

Additionally, other agents may be added to a composition along with Tβ4 or a Tβ4 isoform. Such agents include angiogenic agents, growth factors, agents that direct differentiation of cells, agents that promote migration of cells and agents that stimulate the provision of extracellular matrix material in tissue. For example, and not by way of limitation, Tβ4 or a Tβ4 isoform alone or in combination can be added in combination with any one or more of the following agents: VEGF, KGF, FGF, PDGF, TGFβ, IGF-1, IGF-2, IL-1, prothymosin α and thymosin α1 in an effective amount.

The actual dosage or reagent, formulation or composition that heals damage associated with radiation damage may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

Suitable formulations include the inventive composition at a concentration within the range of about 0.001-10% by weight, more preferably within the range of about 0.005-0.1% by weight, most preferably about 0.01-0.05% by weight.

The therapeutic approaches described herein involve various routes of administration or delivery of reagents or compositions comprising the Tβ4 or other compounds of the invention, including any conventional administration techniques (for example, but not limited to, topical administration, local injection, inhalation, systemic or enteral administration), to a subject. The methods and compositions using or containing Tβ4 or other compounds of the invention may be formulated into pharmaceutical compositions by admixture with pharmaceutically acceptable non-toxic excipients or carriers.

The invention includes use of antibodies which interact with Tβ4 peptide or functional fragments thereof. Antibodies which include pooled monoclonal antibodies with different epitopic specificities, as well as distinct monoclonal antibody preparations are provided. Monoclonal antibodies are made from antigen containing fragments of the protein by methods well known to those skilled in the art as disclosed in PCT/US99/17282, supra. The term antibody as used in this invention is meant to include monoclonal and polyclonal antibodies.

In one embodiment, the invention provides a method for treatment or prevention of damage due to ionizing radiation exposure comprising administering to a subject in need of such treatment, an effective amount of a composition comprising a radiation damage-inhibiting polypeptide comprising amino acid sequence LKKTET, or a conservative variant thereof having radiation damage-inhibiting activity.

In one embodiment, the invention provides a method for treatment or prevention of damage due to ionizing radiation exposure in a subject by contacting tissue with a radiation damage-inhibiting amount of a composition which contains Tβ4 or a Tβ4 isoform. The contacting may be topically, enterally or systemically. Examples of topical administration include, for example, contacting skin or other tissue with a lotion, salve, gel, cream, paste, spray, suspension, dispersion, hydrogel, ointment, or oil comprising Tβ4, alone or in combination with at least one agent that enhances Tβ4 penetration, or delays or slows release of Tβ4 peptides into the area to be treated. Systemic administration includes, for example, intravenous, intraperitoneal, intramuscular or subcutaneous injections, or inhalation (orally or nasally), transdermal, suppository, enema or oral administration of a composition containing Tβ4 or a Tβ4 isoform, etc. A subject may be a mammal, preferably human.

The invention provides a method for the prevention and/or healing and reversal of the body, bodily tissues, and organs and/or symptoms associated therewith, resulting from X-rays, gamma-rays or other forms of ionizing radiation and radiotherapy of (i) cancer patients, (ii) patients receiving radiation or photo-therapy for skin or other disorders, or (iii) individuals exposed to acute or lethal doses of ionizing radiation, by the application of a therapeutically effective amount of a composition comprising Tβ4, Tβ4 analogues, isoforms, or peptide fragments with the amino acid sequence LKKTET and conservative variants thereof.

A method of the invention involves applying a therapeutically effective amount of the composition to a site or systemically on a continuous or periodic basis during a course of therapy to reduce the effects of exposure to ionizing radiation. The duration of administration can range from a single administration to administration for the life of the subject. Preferred courses of administration are in a range of about 1-6 months. Administration can be periodic or continuous. During a course of administration, a composition in accordance with the invention may be administered once, twice, or three or more times per day, and can be administered daily, every other day, every third day, etc.

According to one embodiment, radiation is administered to a target area of a subject, and a composition in accordance with the invention is administered before, during and/or after administration of the radiation to the target area, so as to inhibit radiation damage in an area of said subject outside said target area.

A method of the invention involves utilization of a composition which contains an agent that stimulates the production of LKKTET or Tβ4 or variants thereof or some other actin-sequestering or anti-inflammatory compound.

In one aspect of the method, the healing polypeptide is Tβ4 or an isoform or oxidized form of Tβ4, or a spliced-variant form of Tβ4 in a gel, cream, paste, lotion, spray, suspension, dispersion salve, hydrogel or ointment formulation.

In another aspect of the method the composition may be delivered systemically by injection, orally, nasally, transdermally or any other means.

The composition may be naturally derived or produced using recombinant methodologies, or other synthetic means such as, but not limited to, solid-phase and solution-phase synthesis.

One method includes treating exposure to ionizing radiation or other types of radiation in a subject, comprising administering to the subject a composition containing an agent that regulates the actin-sequestering peptide, LKKTET, or Tβ4 activity. The agent may be an antibody. The antibody may be polyclonal or monoclonal.

One method includes ameliorating the toxicity of radiotherapy comprising treating a subject exposed to such radiotherapy with an agent that regulates Tβ4 activity.

In some embodiments, the Tβ4 regulating agent is an antisense form or other type of antagonist of Tβ4 peptide, or other suitable composition.

The invention may permit significantly increasing the amount of radiotherapy that a cancer patient can receive by administering an effective dose of Tβ4, or Tβ4 analogues, isoforms, or other molecules described herein, containing the amino acid sequence LKKTET and other conservative variants that reduce inflammation, and/or actin toxicity, and/or stimulate angiogenesis and protect radio-sensitive stem cells in the blood, bone marrow, gastrointestinal tract and/or other parts of the body.

1. A method of treatment or prevention of damage due to radiation exposure comprising administering to a subject in need of such treatment an effective amount of a s composition comprising 1) a compound including a radiation damage-inhibiting polypeptide comprising amino acid sequence LKKTET, a conservative variant of LKKTET, an actin-sequestering agent, an anti-inflammatory agent; 2) an agent which stimulates production of said compound in said subject; 3) an agent which regulates said compound in said subject; or 4) an antagonist of said compound, so as to inhibit radiation damage in said subject.

2. The method of claim 1 wherein said compound comprises a polypeptide comprising amino acid sequence LKKTET, or a conservative variant thereof.

3. The method of claim 1 wherein said polypeptide comprises amino acid sequence KLKKTET or LKKTETQ, Thymosin β4 (Tβ4), an N-terminal variant of Tβ4, a C-terminal variant of Tβ4, an isoform of Tβ4, a splice-variant of Tβ4, oxidized Tβ4, Tβ4 sulfoxide, lymphoid Tβ4 or pegylated Tβ4.

4. The method of claim 1 wherein said compound is thymosin beta 4 (Tβ4).

5. The method of claim 1 wherein said compound is present in a gel, cream, paste, lotion, spray, salve, suspension, dispersion, hydrogel or ointment.

6. The method of claim 1 wherein said compound is delivered systemically to said subject by injection, infusion, pulmonary delivery, or orally, rectally, nasally, transdermally, or a combination thereof.

7. The method of claim 1 wherein said agent is an antibody.

8. The method of claim 1 wherein said antagonist is an anti-sense form of said compound.

9. The method of claim 1 comprising administering said compound to said subject so as to protect radiosensitive stem cells in said subject.

10. The method of claim 1 wherein said stem cells are in blood, bone marrow or gastrointestinal tract tissue of said subject.

11. The method of claim 1 wherein said composition is administered systemically.

12. The method of claim 1 wherein said composition is administered topically.

13. The method of claim 1 wherein said composition is administered enterally.

14. The method of claim 1 wherein said radiation is ionizing radiation.

15. The method of claim 1 further including a step of administering radiation to a target area of said subject so as to treat cancer or a tissue abnormality in said target area, wherein said composition is administered to said subject before, during or after administration of said radiation to said target area, or a combination thereof, so as to inhibit radiation damage in said subject outside said target area.

16. The method of claim 15 wherein said composition prevents induced apoptosis of cells of said subject outside said target area.

17. The method of claim 3 wherein said composition is contained in a formulation at a concentration within a range of about 0.001-10% by weight for administration to said subject.

18. A substance for use in manufacturing a medicament for treatment or prevention of damage due to ionizing radiation exposure, comprising 1) a compound including a radiation damage-inhibiting amino acid sequence LKKTET, a conservative variant of LKKTET, an actin-sequestering agent, an anti-inflammatory agent; 2) an agent which stimulates production of said compound in said subject; 3) an agent which regulates said compound in said subject; or 4) an antagonist of said compound, so as to inhibit radiation damage in said subject.
